(19) United States
(12) Patent Application Publication
(10) Pub. No.: US 2021/0038251 A1
(43) Pub. Date: Feb. 11, 2021

(54) DEVICE FOR VISIBLY PUNCTURING ANIMAL TISSUE OR ORGAN

(71) Applicant: Wuhan Youcare Technology Co., Ltd., Wuhan (CN)

(72) Inventors: Jianxing LI, Wuhan (CN); Jinping LI, Wuhan (CN); Chengpeng LIU, Wuhan (CN); Gang LONG, Wuhan (CN); Yeyun MAO, Wuhan (CN); Xuecheng HU, Wuhan (CN)

(21) Appl. No.: 17/080,881

(22) Filed: Oct. 27, 2020

Related U.S. Application Data
(63) Continuation-in-part of application No. PCT/CN2018/104294, filed on Sep. 6, 2018.

(30) Foreign Application Priority Data
Apr. 27, 2018 (CN) 201810395297.4

(51) Int. Cl. A61B 17/34; A61B 1/04

(52) U.S. Cl. CPC A61B 17/3415; A61B 17/3421; A61B 2503/40; A61B 2217/007; A61B 2017/3413; A61B 1/04

(57) ABSTRACT

A device for visibly puncturing an animal tissue or organ including a balloon, a canula, a needle tube, an image acquisition device, and a fluid injection tube. The balloon includes two ends. The canula includes a first axial through hole. The needle tube includes a second axial through hole and is disposed in the first axial through hole. The image acquisition device is disposed in the second axial through hole. The canula is a tubular structure slidable along the axial direction of the needle tube. The balloon is attached to the canula along the axial direction of the canula; the two ends of the balloon are disposed on the outer wall of the canula. The second axial through hole includes a fluid channel disposed between the outer wall of the needle tube and the inner wall of the balloon.

### CROSS-REFERENCE TO RELATED APPLICATIONS

This application is a continuation-in-part of International Patent Application No. PCT/CN2018/104294 with an international filing date of Sep. 6, 2018, designating the United States, now pending, and further claims foreign priority benefits to Chinese Patent Application No. 201810395297.4 filed Apr. 27, 2018. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

The disclosure relates to a device for visibly puncturing an animal tissue or organ.

A conventional device for puncture of an animal tissue or organ comprises a puncture needle, a guide wire, and a sheath. When in use for puncture of tissue or organ of a patient, the puncture needle is first guided to, for example, renal pelvis or calyces of the patient, and then the needle core of the puncture needle is withdrawn and checked whether there is urine on it. If not, the puncture operation is repeated. If so, the needle core is removed and replaced by the sheath, and the guide wire is introduced to the operation site through the sheath. Thereafter, the guide wire is stabilized, the sheath is withdrawn, and a balloon is introduced to the renal pelvis or calyces of the patient along the guide wire. The balloon is filled with normal saline via an external water injection tube and an external syringe. The use of the device is time-consuming, inefficient, and costly.

### SUMMARY

The disclosure provides a device for visibly puncturing an animal tissue or organ, the device comprising a balloon, a canula, a needle tube, an image acquisition device, and a fluid injection tube. The balloon comprises two ends, each end comprising a hole. The canula comprises a first axial through hole. The needle tube comprises a second axial through hole and is disposed in the first axial through hole. The image acquisition device is disposed in the second axial through hole. The canula is a tubular structure slidable along an axial direction of the needle tube. The balloon is attached to the canula along an axial direction of the canula; the two ends of the balloon are disposed on an outer wall of the canula. The second axial through hole comprises a fluid channel disposed between the outer wall of the needle tube and an inner wall of the balloon; and the fluid injection tube is directly connected to the fluid channel.

In a class of this embodiment, the device further comprises a water injection tube communicating with the second axial through hole; the image acquisition device comprises a first outer wall facing the water injection tube and a second outer wall back to back with the first outer wall; a first channel is disposed between an inner wall of the needle tube and the first outer wall of the image acquisition device; and the first channel communicates with the water injection tube.

In a class of this embodiment, the device further comprises an instrument delivery pipe communicating with the second axial through hole and facing the second outer wall; he water injection tube and the instrument delivery pipe are disconnected in the second axial through hole; a second channel is disposed between the inner wall of the needle tube and the second outer wall of the image acquisition device; and the second channel communicates with the instrument delivery pipe.

In a class of this embodiment, the device further comprises a handle; the rear end of the needle tube are fixed in the handle; the handle comprises a cylindrical holder and a tapered holder coaxially fixed on the cylindrical holder; a transition section of the cylindrical holder and the tapered holder comprises a clamping part; and the tapered holder comprises a rear end having an annular circumferential surface.

In a class of this embodiment, the fluid injection tube comprises a first rear end comprising a first joint for fluid injection; the water injection tube comprises a second front end fixed in the handle, and a second rear end comprising a second joint for water injection; the instrument delivery pipe comprises a third front end fixed in the handle, and a third rear end comprising a third joint for instrument introduction; and the rear end of the needle tube is connected to a fourth joint communicating with the image acquisition device.

In a class of this embodiment, the image acquisition device comprises a first end fixed on the inner wall of the needle tube, and a second end passing through the fourth joint.

In a class of this embodiment, the cylindrical holder comprises a front end provided with a threaded hole, and the canula comprises a rear end comprising a threaded section corresponding to the threaded hole.

In a class of this embodiment, the balloon comprises a first welding part and a second welding part respectively disposed on the two ends thereof and fixed on the outer wall of the canula.

In a class of this embodiment, the front end of the needle tube comprises a tapered head; the tapered head comprises a tapered front end and a flat rear end; the tapered head further comprises a central hole communicating with the second axial through hole and the central hole has the same diameter as the second axial through hole; an outer diameter of the flat rear end of the tapered head is no less than an outer diameter of one end of the balloon fixed on the canula; the conical head comprises a spiral step; and a radial section of the balloon is circular, and an axial section of the balloon comprises a rectangular center and two circular ends.

In a class of this embodiment, the device further comprises a sheath disposed on the balloon; the sheath comprises a third axial through hole; when the balloon is filled with a fluid and expanded to show a maximum section width, and the maximum section width is smaller than an inner diameter of the sheath.

### BRIEF DESCRIPTION OF DRAWINGS

FIG. 1 is a top view of a device for visibly puncturing according to one embodiment of the disclosure;

FIG. 2 is a sectional view taken from line A-A in FIG. 1;

FIG. 3 is a local enlarged view of part A in FIG. 2;

FIG. 4 is a local enlarged view of part B in FIG. 2;

FIG. 5 is a local enlarged view of part C in FIG. 2; and

FIG. 6 is a local enlarged view of part D in FIG. 2.

In the drawings, the following reference numbers are used: 1. Balloon; 2. Canula; 3. First joint; 4. Needle tube; 4.1. Tapered head; 5. Image acquisition device; 6. Second axial through hole; 7. Water injection tube; 8. Second joint; 9. Fourth joint; 10. Handle; 10.1. Cylindrical holder; 10.2. Tapered holder; 11. Fluid injection tube; 12. Fluid channel; 13. First channel; 14. Clamping part; 15. Annular circumferential surface; 16. First welding part; 17. Second first welding part; 18. Threaded hole; 19. Sheath; 20. Treaded section; 21. Spiral step; 22. Instrument delivery pipe; 23. Third joint; 24. Second channel.

### DETAILED DESCRIPTION

To further illustrate the disclosure, embodiments detailing a device for visibly puncturing an animal tissue or organ are described below. It should be noted that the following embodiments are intended to describe and not to limit the disclosure.

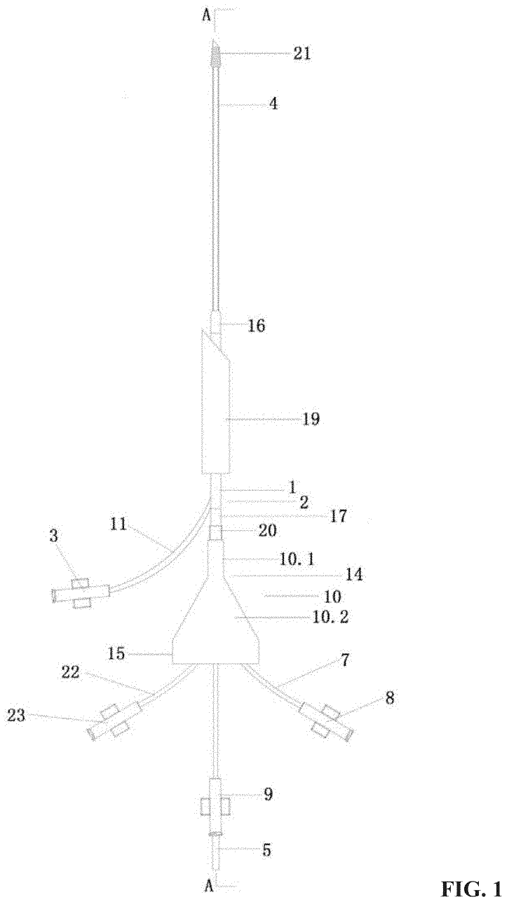

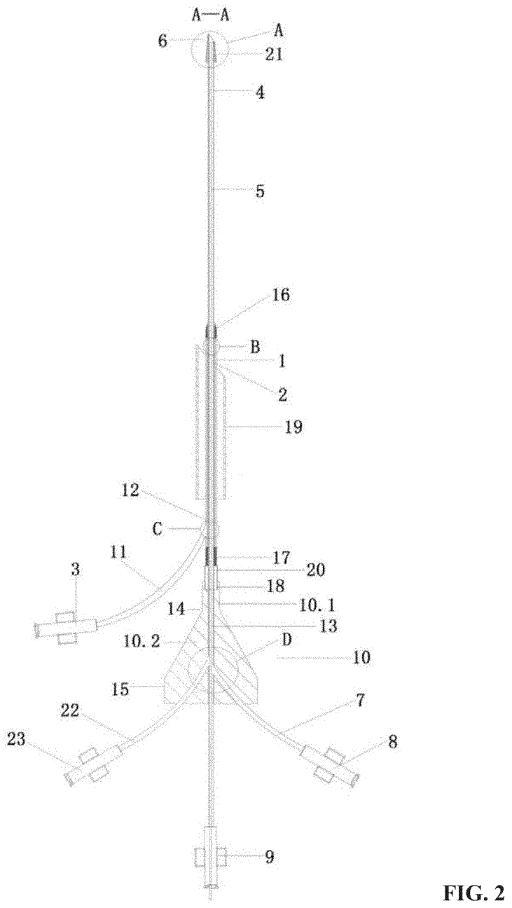

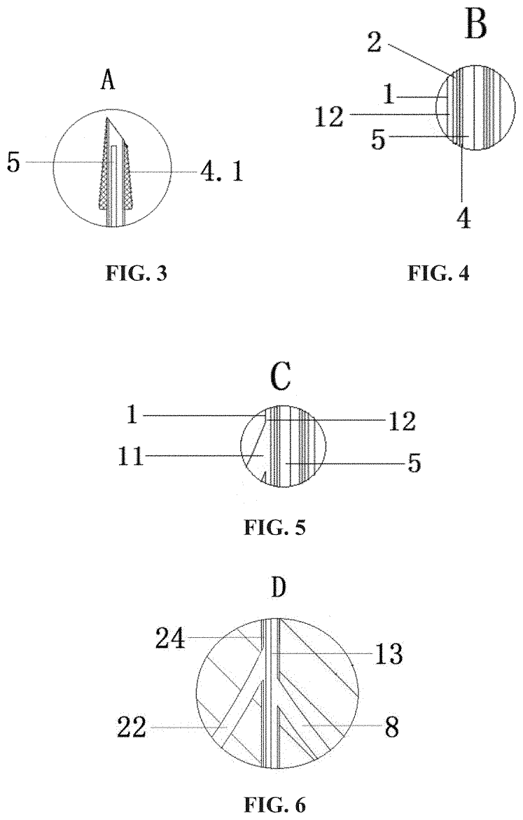

As shown in FIGS. 1-6, the disclosure provides a device for visibly puncturing an animal tissue or organ comprising a balloon 1, a canula 2, and a water inlet tube (a fluid injection tube 11). The canula 2 comprises a first axial through hole. A needle tube 4 is disposed in the first axial through hole of the canula 2. The needle tube 4 comprises a second axial through hole 6. The front end of the needle tube comprises a tapered head 4.1; the tapered head 4.1 comprises a tapered front end and a flat rear end; the tapered head further comprises a central hole communicating with the second axial through hole 6 and the central hole has the same diameter as the second axial through hole 6; the outer diameter of the flat rear end of the tapered head 4.1 is larger than the outer diameter of the end of the balloon fixed on the canula 2 (the flat rear end of the tapered head 4.1 is configured to limit the position of the front end of the canula 2); the conical head 4.1 comprises a spiral step 21. The spiral step 21 can enhance the echo of B-ultrasound and display its position on the display screen, which increases the guidance and accuracy of the puncture operation. An image acquisition device 5 is disposed in the second axial through hole 6. The device further comprises a handle 10; the rear end of the needle tube 4 are fixed in the handle 10; the handle 10 comprises a cylindrical holder 10.1 and a tapered holder 10.2 coaxially fixed on the cylindrical holder 10.1; the transition section of the cylindrical holder 10.1 and the tapered holder 10.2 comprises a clamping part 14 shaped like a human's finger (this is convenient for a user to hold the device); and the tapered holder 10.2 comprises a rear end having an annular circumferential surface 15 (this is convenient for the operator to hold, increasing the friction force of hands, thus ensuring the stable operation of the medical staff). The cylindrical holder 10.1 comprises a front end provided with a threaded hole 18, and the canula 2 comprises a rear end comprising a threaded section 20 corresponding to the threaded hole. The rear end of the needle tube 4 is connected to the fourth joint 9 communicating with the image acquisition device (a fiber scope, for example). The front end of the fiber scope is extended into the second axial through hole 6 and the rear end thereof passes through the fourth joint 9. A first channel 13 is disposed between an inner wall of the needle tube and the outer wall of the image acquisition device 5; and the first channel 13 communicates with a water injection tube 7. The front end of the water injection tube 7 is fixed in the handle 10, and the rear end thereof passes through the handle and is connected to a second joint 8 for water injection. An instrument delivery pipe 22 (a guide wire pipe, for example) is connected to the second axial through hole 6. The water injection tube 7 and the instrument delivery pipe 22 are disconnected in the second axial through hole. A second channel 24 is disposed between the inner wall of the needle tube and the outer wall of the image acquisition device 5 to allow the instrument delivery pipe to pass through. The front end of the guide wire pipe is fixed in the handle 10, and the rear end thereof is connected to a third joint 23 for instrument introduction.

The radial section of the balloon is circular, and an axial section of the balloon comprises a rectangular center and two circular ends. The balloon 1 comprises a first welding part 16 and a second welding part 17 respectively disposed on the two ends thereof and fixed on the outer wall of the canula 2. A fluid channel 12 is disposed between the outer wall of the needle tube 4 and the inner wall of the balloon 1. A fluid injection tube 11 is directly connected to the fluid channel. The fluid injection tube 11 comprises a first joint 3 for fluid injection. The device further comprises a sheath 19 disposed on the balloon; the sheath 19 comprises a third axial through hole; when the balloon 1 is filled with a fluid and expanded to show a maximum section width, and the maximum section width is smaller than an inner diameter of the sheath.

In certain embodiments, the fiber scope (the image acquisition device) is integrated with or independent from the needle tube 4. When the fiber scope is independent from the needle tube, the fiber scope is inserted into the needle tube 4 via the fourth joint 9 to observe the puncture position, and pull out after the operation.

The usage method of the device for visibly puncturing an animal tissue or organ is described as follows:

1) The fiber scope is inserted into the second axial through hole 6 and locked in a target location. The sheath 19 is disposed on the needle tube 4 (if the fiber scope is integrated with the needle tube 4, only need to dispose the sheath on the needle tube 4). The sheath 19 is a hollow pipe; when the balloon 1 is filled with a fluid and expanded to show a maximum section width, and the maximum section width of the balloon 1 is smaller than the inner diameter of the sheath 19.

2) The optical fiber of the fiber scope is connected to a camera system until a normal image can be seen on the display.

3) The device for visibly puncturing an animal tissue or organ is held by hand. Under B-ultrasound equipment, the needle tube 4, the balloon 1, and canula 2 cooperate with each other for puncture. The needle tube 4 reaches the renal pelvis and renal calices through the epidermis. The renal collection system can be seen on the optical fiber mirror. If the imaging is not clear, a small amount of physiological saline can be injected into the needle tube 4 through the second joint 8 to clear the vision so as to determine the puncture position of the renal collection system.

4) When the needle tube 4 reaches the target position, rotate the threaded section 20 until the threaded section 20 is detached from the threaded hole 18. The canula 2 is moved back and forth to the expanded position. Normal saline is injected into the balloon 1 through a water filling connector to expand the balloon 1 using a syringe. When the pressure on the syringe reaches the set pressure (25 atmospheres), stop filling water and hold for 30 seconds. The sheath 19 is pushed forward along the needle tube 4 and sheathed on the balloon 1. Thereafter, the water in the balloon 1 is drained and the balloon is taken out. Thus, the sheath 19 stays in an expanded channel completed by the balloon 1, so that the expanded state is remained, that is to say, the working channel of percutaneous renal surgery is established.

In 3), when the needle tube 4 reaches the target position, the guide wire is introduced into the second axial through hole 6 via the third joint 23 and the instrument delivery pipe 22. After 4) is completed, only the sheath 19 and the guide wire are remained, and other parts are pulled out. The guide wire is used for introduction of subsequent surgical instruments.

The device for visibly puncturing an animal tissue or organ incorporates the needle tube 4 and the balloon 1, and the fiber scope is built-in in the device or introduced temporarily as needed. Using the device, the puncture position can be clearly observed thus improving the operation accuracy. Compared with a convention puncture device which involves the withdrawal of the puncture needle, the placement and stabilization of the guide wire, the withdrawal of the sheath of the puncture needle, the expansion of the sheath core along the guide wire, and the withdrawal of the sheath core, the operation of the device of the disclosure is simple. The operations of the puncture and the expansion are fulfilled in the same device, without the operations of withdrawal or replacement of the guide wire, thus saving the usage of consumable items, reducing the operation time, reducing the risk of surgery, reducing the cost of surgery, and reducing the pain of patients. During the expansion process, the state of the renal collection system can be monitored through the fiber optic mirror to reduce the risk of bleeding. The balloon 1 can be delivered to an optimal expansion position by adjusting the position of the canula 2 back and forth thus ensuring the smooth operation. The device of the disclosure can be applied in general surgery, neurosurgery, urology, hepatobiliary surgery, gynecology and so on.

It will be obvious to those skilled in the art that changes and modifications may be made, and therefore, the aim in the appended claims is to cover all such changes and modifications.

What is claimed is:

1. A device, comprising:
a balloon, the balloon comprising two ends, each end comprising a hole;
a canula comprising a first axial through hole;
a needle tube comprising a second axial through hole, the needle tube being disposed in the first axial through hole;
an image acquisition device disposed in the second axial through hole; and
a fluid injection tube;
wherein:
the canula is a tubular structure slidable along an axial direction of the needle tube;
the balloon is attached to the canula along an axial direction of the canula;
the two ends of the balloon are disposed on an outer wall of the canula;
the second axial through hole comprises a fluid channel disposed between the outer wall of the needle tube and an inner wall of the balloon; and
the fluid injection tube is directly connected to the fluid channel.

2. The device of claim 1, further comprising a water injection tube communicating with the second axial through hole; wherein the image acquisition device comprises a first outer wall facing the water injection tube and a second outer wall back to back with the first outer wall; a first channel is disposed between an inner wall of the needle tube and the first outer wall of the image acquisition device; and the first channel communicates with the water injection tube.

3. The device of claim 2, further comprising an instrument delivery pipe communicating with the second axial through hole and facing the second outer wall; wherein the water injection tube and the instrument delivery pipe are disconnected in the second axial through hole; a second channel is disposed between the inner wall of the needle tube and the second outer wall of the image acquisition device; and the second channel communicates with the instrument delivery pipe.

4. The device of claim 3, further comprising a handle; wherein a rear end of the needle tube are fixed in the handle; the handle comprises a cylindrical holder and a tapered holder coaxially fixed on the cylindrical holder; a transition section of the cylindrical holder and the tapered holder comprises a clamping part; and the tapered holder comprises a rear end having an annular circumferential surface.

5. The device of claim 4, wherein the fluid injection tube comprises a first rear end comprising a first joint for fluid injection; the water injection tube comprises a second front end fixed in the handle, and a second rear end comprising a second joint for water injection; the instrument delivery pipe comprises a third front end fixed in the handle, and a third rear end comprising a third joint for instrument introduction; and the rear end of the needle tube is connected to a fourth joint communicating with the image acquisition device.

6. The device of claim 5, wherein the image acquisition device comprises a first end fixed on the inner wall of the needle tube, and a second end passing through the fourth joint.

7. The device of claim 4, wherein the cylindrical holder comprises a front end provided with a threaded hole, and the canula comprises a rear end comprising a threaded section corresponding to the threaded hole.

8. The device of claim 1, wherein the balloon comprises a first welding part and a second welding part respectively disposed on the two ends thereof and fixed on the outer wall of the canula.

9. The device of claim 1, wherein a front end of the needle tube comprises a tapered head; the tapered head comprises a tapered front end and a flat rear end; the tapered head further comprises a central hole communicating with the second axial through hole and the central hole has the same diameter as the second axial through hole; an outer diameter of the flat rear end of the tapered head is no less than an outer diameter of one end of the balloon fixed on the canula; the conical head comprises a spiral step; and a radial section of the balloon is circular, and an axial section of the balloon comprises a rectangular center and two circular ends.

10. The device of claim 1, further comprising a sheath disposed on the balloon; wherein the sheath comprises a third axial through hole; when the balloon is filled with a fluid and expanded to show a maximum section width, and the maximum section width is smaller than an inner diameter of the sheath.
